(19) United States
(12) Patent Application Publication
(10) Pub. No.: US 2006/0089411 A1
(43) Pub. Date: Apr. 27, 2006

(54) Treatment of Stargardt's disease and other lipofuscin disorders with combined retinaldehyde inhibitor and zeaxanthin

(76) Inventor: Dennis L. Gierhart, Chesterfield, MO (US)

Correspondence Address: Patrick D. Kelly, 11939 Manchester #403, St. Louis, MO, 63131, US

(21) Appl. No.: 11/198,114

(22) Filed: Aug. 6, 2005

Related U.S. Application Data
(60) Provisional application No. 60/599,729, filed on Aug. 7, 2004.

(51) Int. Cl. A61K 31/203; A61K 31/12; A61K 31/015

(52) U.S. Cl. 514/559; 514/690; 514/763

(57) ABSTRACT

Zeaxanthin, a natural carotenoid, can improve and increase the ability of enzyme inhibitors that can slow down certain enzymes that are contributing to toxic metabolite accumulation in people who suffer from Stargardt's disease or other lipofuscin disorders. Such enzyme inhibitors include retinoid analogs such as isotretinoin, commonly known by the trademark, ACCUTANE. This drug binds to and inhibits at least two retinal enzymes, known as RPE65 and short chain dehydrogenase, which create surplus metabolites that feed into a pathway that eventually creates toxic metabolites in people with Stargardt's disease. However, isotretinoin treatment alone is not highly effective; therefore, use of zeaxanthin as an adjunctive treatment can improve the efficacy and outcomes of such treatments.

### RELATED APPLICATION

This application claims the benefit under 35 USC 119(e) of provisional application 60/599,729, filed on Aug. 7, 2004.

### FIELD OF THE INVENTION

This invention is in the field of pharmacology and ophthalmology, and relates to compounds that can help preserve vision in people who suffer from eye and vision disorders (including Stargardt's disease) that involve retinal accumulation of unwanted material called lipofuscin.

### BACKGROUND OF THE INVENTION

Stargardt's disease is a known genetic disorder that severely damages the vision, and almost always leads to functional blindness. Stargardt's victims usually begin suffering from serious and then severe vision problems in late childhood or their teenage years. It can be retarded somewhat by using sunglasses and various sunlight avoidance techniques to reduce the amount of blue, ultraviolet (UV), and near-UV radiation that enters the eyes. The damage occurs in the retina, and mainly affects the yellow-colored center portion of the retina, called the macula.

Most cases of Stargardt's disease involve a defect in a gene that encodes a protein in the “ATP-binding cassette” (ABC) family of proteins. Because numerous ABC proteins are known, and because the particular protein involved in Stargardt's disease can be regarded as a receptor or “rim” protein, the particular ABC protein that is defective or missing in Stargardt's disease is usually referred to as the ABCR or abcr protein. Some articles also refer to that same protein as the ABC-A4 or abca4 protein.

Many people have one defective copy of the ABCR gene, along with one properly functioning copy. This combination is known as the ± genotype, which indicates one functional copy, and one missing or defective copy. If someone has at least one properly functioning copy of the gene, they will not suffer from the most common form of Stargardt's disease; therefore, the defective gene is referred to as a “recessive” gene. If both parents have the ABCR ± genotype, there is a 25% chance that a child will inherit the −/− genotype, and will not have any properly functioning copies of the ABCR gene or proteins. If this happens, the recessive disease is fully manifested, and the person will suffer from Stargardt's disease. Under all currently known and available forms of treatment, the disease will lead eventually to severe damage to the vision, usually leading to functional blindness.

It also should be noted that (i) a different form of Stargardt's disease can be caused by a single dominant gene; and, (ii) people who carry the ABCR ± genotype (with only half the normal supply of the protein) tend to suffer from various eye problems, including excessive lipofuscin accumulation, some degree of retinal deterioration, and elevated risks of age-related macular degeneration, but those problems stop short of full-blown Stargardt's disease. Those matters are discussed in more detail below.

The problem that leads to retinal damage, in the main class of Stargardt's disease that involves the recessive ABCR −/− genotype, involves the processing and gradual accumulation of certain metabolites, in certain cell types in and around the retina.

One key intermediate is all-trans-retinaldehyde (also called at-retinal, trans-retinal, etc.). This compound occurs in everyone, and it is essential in the chemical reactions used by retina cells to convert light into nerve impulses. It is formed when dehydrogenase enzymes convert the hydroxy group at the end of Vitamin A (also called retinol) into an aldehyde group.

After trans-retinal has been formed, it goes back and forth between a trans isomer and a cis isomer (called 11-cis-retinal), which are illustrated in FIG. 1, which are prior art. The structural difference between these two isomers is that the trans isomer is relatively straight (more precisely, it zig-zags back and forth in a regular and consistent way, which leads to an overall structure that is generally linear), while the cis isomer has a kink or bend in its chain, because of a different bond arrangement that starts at the #11 carbon atom, as shown in FIG. 1.

Because of certain ways that atoms and electrons want to be separated from each other, the kinked cis isomer has a slightly more crowded, compressed, and stressed structure, which involves a slightly higher energy level than the straight and relatively relaxed trans isomer. Therefore, a relaxed and low-energy trans isomer can be converted into a stressed and higher-energy cis isomer only by means of a significant energy input, which requires an enzyme reaction to occur. By contrast, when the higher-energy cis isomer is hit by a light photon, that single photon of light can give it enough of a “nudge” to cause it to fall off of its higher energy plateau, and drop back to the more relaxed, lower-energy trans structure.

This very slight energy difference between the higher-energy cis isomer and the lower-energy trans isomer makes those two isomers ideal for cycling back and forth, countless times, in the chemical pathway that allows light to be converted into nerve signals. After an enzyme converts a low-energy trans isomer into a high-energy cis isomer, the cis isomer associates with a protein called opsin, to form rhodopsin, which is the light-sensitive compound that plays a crucial role in vision. When a molecule of rhodopsin is hit by a light photon, the cis isomer converts back to the relaxed trans isomer, and it detaches from the opsin protein. This electrochemical reaction creates a small voltage surge, which is processed by the retinal neuron into a nerve impulse, which is sent to the brain, for processing. The trans isomer is then processed and handled by enzymes, in ways that convert it back into the cis isomer, which can once again associate with an opsin molecule to form rhodopsin, to complete the cycle. This cyclic process, and the molecules and cells involved, are well-known, and are described and illustrated in numerous reference works.

In most people, all-trans-retinal is handled efficiently, in ways that prevent it from accumulating to levels that can cause problems. However, a relatively small portion of the system that handles all-trans-retinal requires the involvement of the ABCR protein, which is defective in Stargardt patients. Since Stargardt patients don't have that protein, a surplus of all-trans-retinal will gradually accumulate over a span of years, in their retinas and surrounding tissues. Eventually, as described in articles such as Radu et al 2003, in the retinas of Stargardt patients, some portion of that surplus all-trans-retinal will trickle through a multi-step pathway that leads to a toxic metabolite called N-retinylidene-N-retinyl-ethanolamine, which is commonly called A2E since it has two segments from all-trans-retinaldehyde, coupled to an ethanolamine ring. It takes more than a decade to accumulate to toxic levels, and it occurs mainly in the “retinal pigmented epithelium” (RPE) layer, a layer of darkly-pigmented cells positioned behind the retina.

Eventually, A2E becomes toxic to the RPE cells. At least four possible mechanisms for the toxicity are discussed in the literature, and any or all of them may be involved in causing cytotoxic damage, at varying levels, in different people. Those four postulated mechanisms are: (1) interference with cytochrome oxidase enzymes, which perform useful and essential roles handling and removing waste products; (2) formation of compounds called epoxides and/or oxiranes, which contain oxygen atoms in stressed ring structures that can break in ways that will form unstable radicals, which will attack and damage proteins, DNA, and cell membranes; (3) damage to lysosomes, the acidic organelles that cells use to digest and metabolize various types of molecules; and, (4) because of its shape and structure, A2E may act as a “surfactant”, comparable to a detergent that can disrupt and create holes or leakage in the membranes of cells and organelles.

As noted above, the A2E toxin in a Stargardt's patient accumulates mainly in the RPE layer, directly behind the retina, and much of the damage caused by A2E occurs in the RPE layer. Since that layer is essential to good vision, the person will begin noticing a loss of clear vision, usually between the ages of 10 and 20. A retinal examination will be performed, and it will reveal abnormally large quantities of a material called lipofuscin, in and behind the retina. Lipofuscin is formed mainly from the debris of dead cells, and in patients who suffer from abnormal lipofuscin accumulation, it usually contains a significant quantity of the A2E toxin (the formation and accumulation of lipofuscin, in such patients, may result from an effort by the cells to coat, sequester, and inactivate the A2E toxin, in a process called “entombment”). Lipofuscin that contains A2E is fluorescent (i.e., it will emit a fluorescent wavelength, when a diagnostic light having a different excitatory wavelength is shown into the eye); as such, it is often called a “fluorophore”.

Since fluorescence allows lipofuscin that contains A2E to be readily seen or photographed during a non-invasive examination, and since it rarely occurs in any significant quantity in people below the age of about 50 or 60, its presence in substantial quantities during a retinal examination of a patient under the age of 20 provides a fairly reliable indication that the young person has Stargardt's disease. If desired, a genetic analysis can be carried out, to determine the DNA sequence of the defective ABCR gene(s) in that patient.

The foregoing description is merely an overview. Because the defective genes and proteins have been fully sequenced, because their correlation with Stargardt's disease is clear, and because the A2E metabolite has been clearly identified as a cytotoxic agent, Stargardt's disease is one of the most closely and intensively studied diseases among all the retinal or macular diseases, and extensive detail is available in the literature. Articles on the ABCR gene and protein include Sun et al 2001 and Koenekoop 2003. Articles on lipofuscin accumulation, traits, and effects include Delori et al 1995 and Mata et al 2001. Articles on ABCR mutations and the A2E formation pathway include Parish et al 1998, can Driel et al 1998, Holz et al 1999, Mata et al 2000, and Glazer et al 2002. Articles on how A2E causes cell damage and death include Schutt et al 2000, Suter et al 2000, Sparrow et al 2000, 2001, and 2002, and Radu et al 2004. Review articles include Ben-Shabat et al 2001, Donoso et al 2001, Glazer et al 2002, and Wolf 2003, and those reviews cite hundreds of additional articles.

After the role of the ABCR gene defect in Stargardt's disease became known, researchers created a “mouse model” of the disease, by using genetic engineering methods to disrupt, replace, or otherwise “knock out” the ABCR gene in albino mice. Strains of mice carrying the ABCR −/− genotype, with no functioning copies of the ABCR gene or protein, were created, and tests on these mice showed various similarities between their retinal behaviors and problems, and the retinal behaviors and problems observed in humans. Those mouse models are described in articles such as Mata et al 2000 and 2001, and Radu et al 2004.

Cell culture tests are also used to study retinal and macular cells and disorders, and to screen and evaluate candidate drugs that may be able to help slow down or prevent the cytotoxic damage caused by factors such as oxygen radicals, overexposure to blue and/or UV light, and the A2E toxin. These types of tests, described in articles such as Holz et al 1999, Schutt et al 2000, Suter et al 2000, Nilsson et al 2003, and Wrona et al 2003 and 2004, normally use RPE cells that have been grown on solid supports, usually to a “monolayer” in a petri dish or in the wells of a multi-well titer plate; alternately, some in vitro tests use liposomes, micelles, or other membrane structures that can be formed without requiring cells. Some tests use synthetic A2E, made by chemists using published techniques.

Three other points should be noted, which lead to an important result. First, as mentioned above, animals and people that have the ABCR ± genotype (i.e., they inherited a good copy of the gene from one parent, and a defective copy from the other parent), tend to accumulate various unwanted metabolites (including A2E and lipofuscin) at elevated concentrations, in and around their retinas, when compared to people with the ABCR +/+ genotype, who have a full set of properly functioning ABCR genes from both parents.

Second: a number of versions of the ABCR protein have been identified that appear to be partially impaired, but are at least partially functional, when compared to fully functional ABCR proteins in healthy eyes.

Third: the extent of damage that will be caused by missing or non-functional ABCR proteins, or by impaired but partially functional ABCR proteins, also depends on the presence and concentrations of other genes and proteins, which vary substantially among different racial and ethnic groups.

Due to those factors, people who suffer from partial defects in their ABCR genes or proteins often suffer from eye problems that emerge later in life, and that may be diagnosed as retinitis pigmentosa, age-related macular degeneration, rod and/or cone dystrophy, or “Stargardt-like disease”, rather than being formally classified as Stargardt's disease.

It also should be noted that some but not all cases of Stargardt's disease are classified as “fundus flavimaculatus”. That term refers to a condition in which the “fundus” portion of the macula contains visible yellowish-white flecks. Some articles apparently use the two diagnostic terms interchangeably; however, not all cases of Stargardt's disease lead to visible colored flecks in the retina.

In addition, a completely different genetic defect has been discovered to cause a rare form of Stargardt's disease, discussed in articles such as Vrabec et al 2003. This defect involves a gene called ELOVL4, which is involved in the elongation of certain fatty acids. In the ELOVL4 disease (sometimes referred to as Stargardt's Type 3), a single copy of the malfunctioning gene will create the disease; therefore, this version is classified as a dominant gene defect, rather than a recessive gene defect. It was initially classified as a form of Stargardt's disease because the symptoms (including lipofuscin accumulation) and the age of onset tend to be very similar. However, rather than involving the ABCR gene and protein as a primary mechanism, this disorder focuses primarily on fatty acids, and clinical trials are being planned to determine whether the essential fatty acid called DHA (docosahexaenoic acid) may be able to help patients with this type of Stargardt's disease. While it is not known at this time whether the treatments described herein (i.e., zeaxanthin in combination with either or both of a dehydrogenase enzyme inhibitor and/or DHA) will be able to effectively treat the ELOVL4 form of Stargardt's disease, these combined treatments merit expedited evaluation in such patients, since they may be able to help more effectively than any other known treatments.

Accordingly, Stargardt's disease is regarded herein as an archetypal and illustrative example of a class of retinal disorders referred to herein as “lipoftiscin accumulation disorders” (also referred to simply as lipofuscin disorders, since any significant accumulation of lipofuscin is detrimental, and may be a symptom of a serious underling disorder, especially if it occurs in significant quantities in someone less than about 60 years old). It is believed that the combined treatments disclosed herein offer good candidate treatments that should be evaluated for potential benefits and efficacy, in treating any and all disorders involving the accumulation of lipofuscin (and/or the A2E toxin, which contributes to the formation of lipofuscin) in or near retinal tissues.

Isotretinoin (Accutane™) as a Potential Treatment

It has been reported, in Radu et al 2003 and in published U.S. patent application 2003/032,078 (Ser. No. 09/885,303, filed in Jun. 2001 by Dr. Gabriel Travis, the senior author of Radu et al 2003), that a drug called isotretinoin can help slow and reduce the accumulation of lipofuscin, in the retinas of mice that have the genetically engineered ABCR −/− gene defect.

Isotretinoin is commonly known by its trademark, ACCUTANE™. It normally is used to treat acne and complexion problems, mainly in teenagers.

The more informative chemical name for isotretinoin is 13-cis-retinoic acid. As shown at the bottom of FIG. 1, it has the same type of kinked and bent structure as 11-cis-retinal; however, the kink is closer to the end of the chain, beginning at the #13 carbon atom, rather than at the #11 carbon atom as in naturally occurring 11-cis-retinal. This makes the straight-chain portion longer, and more closely similar to all-trans-retinal.

As a result, isotretinoin acts as an “analog” of both all-trans-retinal and 11-cis-retinal. It will bind (with some level of affinity) to some of the same enzymes that bind to either or both of the two natural retinal isomers. However, because isotretinoin does not have the normal and proper structure of either of the natural isomers, it is sometimes difficult for an enzyme that has grabbed it to release it quickly and properly. Therefore, isotretinoin will bind to and inhibit at least two enzymes that are important in vision.

This was discovered, because people who were taking isotretinoin noticed they were suffering from “night blindness”, with decreased acuity and responsiveness of their vision under moderately dark conditions. When researchers studied that condition, they discovered that isotretinoin inhibit at least two different enzymes involved in vision. One of those enzymes, discussed in Radu et al 2003, is a “short chain dehydrogenase” enzyme that converts 11-cis-retinol (the alcohol form, with a hydroxy group at the end) into 11-cis-retinal (which has the —CHO aldehyde group at the end). The other enzyme was discovered somewhat later, and is called RPE65, as discussed in Gollapalli et al 2004.

When those two research teams, studying different enzymes, realized (separately) that isotretinoin can and will slow down the vision cycle by inhibiting certain enzymes, they both recognized that isotretinoin might therefore be useful, in slowing down the gradual damage caused by certain types of retinal and macular disorders. That insight has been further developed, tested, and advanced by Dr. Gabriel Travis, the senior author of Radu et al 2003. As described in US patent application 2003/032,078, using mice having the ABCR −/− knockout genotype, Dr. Travis showed that isotretinoin, if administered to gene-deficient mice in high dosages, was able to slow down the accumulation of A2E, the toxic metabolite that causes damage to the retinas of Stargardt's patients.

That activity was attributed, by Travis, to isotretinoin's ability to inhibit the short chain dehydrogenase enzyme that converts 11-cis-retinol into 11-cis-retinal. However, that effect may also have been due, at least in part, to the additional inhibition of the RPE65 enzyme as well, as identified subsequently by Gollapalli et al 2004.

Accordingly, isotretinoin offers a promising research lead, and a ray of hope, for people suffering from Stargardt's disease and other eye disorders involving abnormal lipofuscin accumulation, and for researchers who are trying to find better ways to treat such disorders.

However, that reported discovery apparently will not and cannot lead directly to an effective treatment for Stargardt's disease in humans, since the dosages and concentrations of isotretinoin that were needed to significantly reduce A2E concentrations in ABCR −/− mice, were many times higher than the highest safe and tolerable levels of isotretinoin that have been approved for use in humans.

As a result, Travis's patent application 2003/032,078 focused not on the use of isotretinoin (ACCUTANE) to treat Stargardt's disease, but on a method of screening other candidate drugs (including analogs of isotretinoin) to evaluate their potential utility for treating macular or retinal degeneration, by evaluating their ability to inhibit the activity of short chain dehydrogenase enzymes.

While that work is promising, it has not reached fruition. To the best of the Applicant's knowledge and belief, as this is being written, no analogs or derivatives have been identified to date that have shown any special or particular promise, above and beyond the level of isotretinoin, in slowing down the formation of lipofuscin and/or the A2E toxin. Instead, people are trying to organize more animal tests of isotretinoin using ABCR −/− mice, and trying to decide whether to test the highest lawfully-allowed dosages of isotretinoin, in teenagers who have been diagnosed with Stargardt's disease.

Upon learning of those efforts, the Applicant herein began considering alternate approaches that might be able to help increase the potency and efficacy of isotretinoin in reducing A2E and/or lipofuscin accumulation, while at the same time reducing any toxic side effects of the drug. Because of a different project (described below) he had been working on (which is not prior art against this current invention), he realized that a certain carotenoid stereoisomer, called 3R,3′R-zeaxanthin, may be ideally suited and highly effective for providing a synergistic benefit that can supplement the potential benefits of isotretinoin or other compounds that may be able to inhibit the short-chain dehydrogenase and/or RPE65 enzymes, in patients suffering from Stargardt's disease or other disorders that involve unwanted accumulation of A2E and/or lipofuscin.

To understand that insight, it is necessary to provide additional background information on carotenoids in general, on zeaxanthin in particular, and on a recent discovery that zeaxanthin can provide protective and beneficial effects, if used to “load up” the retinas of patients before they undergo a type of therapy that uses lasers and phototoxic drugs to kill blood vessels that are growing out of control, in a different type of retinal disorder called “wet” macular degeneration.

Background on Carotenoids

It has been recognized for years that carotenoids play important roles in various retinal functions, and in macular diseases, and they are discussed in detail in numerous textbooks and review articles.

Briefly, carotenoids are large organic molecules with carbon chains that have alternating single and double bonds, as illustrated by the structures of three relevant carotenoids in FIG. 2, which is prior art. In plants and a few types of bacteria, carotenoids are created by coupling together multiple copies of a 5-carbon precursor called isoprene, which has two unsaturated bonds. Therefore, carotenoids can be referred to as isoprenoids, and many of them contain multiples of 5 carbon atoms (for example, beta-carotene, lutein, and zeaxanthin all contain exactly 40 carbon atoms).

Carotenoids absorb light in the ultraviolet (UV), near-UV, and blue portion of the spectrum. Because blue and near-UV wavelengths are absorbed while other wavelengths are reflected and emitted, carotenoids generally appear as red, orange, and yellow pigments. When the leaves of trees or bushes turn red, orange, and yellow in the fall, those colors are due to carotenoids, which become the dominant pigments in the leaves after chlorophyll production stops.

In addition to absorbing UV radiation, which otherwise can be toxic and even lethal to cells, carotenoids are anti-oxidants. They can neutralize and “quench” various types of unstable “radicals” that have unshared electrons (often called free radicals, oxidative free radicals, reactive oxygen species, etc.).

Since oxidative free radicals are often created when UV photons hit and break apart various types of biomolecules, the ability of carotenoids to absorb, neutralize and quench both UV photons, and oxidative free radicals, is an exceptionally useful trait. Therefore, carotenoids evolved over the eons as essential protective compounds in plants, and in some types of microbes that grow in areas exposed to direct sunlight.

There are over 600 known naturally-occurring carotenoids, but only about 20 have been found in human blood or tissues, and only three specific carotenoids are regarded as being truly important, in human eyes. Those three are beta-carotene, lutein, and zeaxanthin.

As shown in FIG. 2, beta-carotene does not contain any oxygen atoms. It is a true hydrocarbon, made entirely of carbon and hydrogen atoms. Therefore, it is non-polar, very oily, and hydrophobic. This causes it to avoid and minimize any contact with water molecules and aqueous fluids. Therefore, when it is ingested by animals, it is deposited mainly into the interior layers of cell membranes, as indicated in FIG. 3.

The most common fate of beta-carotene molecules is that they are broken in half, length-wise, to release two molecules of Vitamin A, also called retinol. The hydroxy group at the end of retinal is then converted (by dehydrogenase enzymes) into an aldehyde group, to form all-trans-retinal. This is the same straight-chain isomer that is shown in FIG. 1; it cycles back and forth with the bent-chain isomer, 11-cis-retinal, in the vision cycle, as described above.

In healthy retinas, that chemical vision cycle uses ABCR proteins to help make sure the lower-energy isomer, all-trans-retinal, is converted back into the higher-energy isomer, 11-cis-retinal. However, as mentioned above, people with Stargardt's disease do not have properly functioning copies of the ABCR protein. Therefore, surplus quantities of all-trans-retinal will gradually accumulate in their retinal cells, especially in cells in the RPE layer. A small portion of the all-trans-retinal that accumulates eventually will be converted into some quantity of A2E, the toxin that damages and kills cells in the RPE layer that supports the retina.

That fact is mentioned again, because it points to a crucially important fact: the dietary source of all-trans-retinal, which gradually accumulates in surplus and unwanted quantities, and which is gradually converted into the A2E toxin that kills RPE cells and destroys the eyesight of people who suffer from Stargardt's disease, is beta-carotene.

Therefore, a strong presumption arises that ingestion of large and heavy dosages of beta-carotene, among people who suffer from Stargardt's disease, is ill-advised and dangerous. Because of how biochemical reactions to try to sustain equilibrium and homeostasis, ingestion of large and heavy dosages of beta-carotene is likely to drive and stimulate the formation of higher quantities of all-trans-retinal. In Stargardt patients, that is a dangerous step, because the formation of higher quantities of all-trans-retinal will in turn drive and promote the accumulation of surplus and unwanted all-trans-retinal, which in turn will boost the production of the A2E toxin, which kills the RPE cells and destroys the eyesight of Stargardt patients.

Some scientists have begun to realize that beta-carotene is not an entirely benevolent or benign carotenoid. Indeed, reports as early as Burton 1984 showed that if high oxygen concentrations are present, beta-carotene actually reverses its useful and beneficial anti-oxidant activities, and becomes a destructive pro-oxidant (i.e., it begins to trigger and accelerate the formation of unstable and destructive oxidative radicals). Those high oxygen concentrations do not exist in most types of organs or tissues; however, they do exist in lungs, which interact directly with oxygen in air that is inhaled. As a result, it has been clearly and repeatedly shown, in large and well-run trials, that high-dosage beta-carotene actually increases the rates and risks of lung cancer, in people with risk factors such as smoking. As a result of those and other problems, some experts have openly and publicly declared that all high-dosage beta-carotene vitamin and dietary supplements should be declared dangerous, and taken off the market.

That warning would appear to be especially true for Stargardt's patients, since the A2E toxin that eventually destroys the RPE layer of their retinas comes from beta-carotene, via the retinol and retinal pathway. By restricting the beta-carotene intake of Stargardt patients to quantities that should be no higher than necessary, it may be possible to slow down the gradual accumulation of surplus all-trans-retinal, and the formation of the A2E toxin.

However, that insight apparently has not been adequately recognized, understood, or appreciated by the scientific and medical experts at the National Eye Institute (NEI), or at the eye care companies that continue to sell products that arose from the AREDS-1 study. As this is being written, two products (OCUVITE PRESERVISION™ pills, sold by Bausch & Lomb, and I-CAPS AREDS™ pills, sold by Alcon) contain high-dosage beta-carotene. Those two products continue to be widely and actively advertised and marketed to anyone suffering from macular degeneration, and they are described as the only products that have ever been clinically proven to slow down or help prevent macular degeneration. While both companies apparently have decided to offer AREDS-1 variants with somewhat lower beta-carotene dosages, for smokers, those companies apparently have not taken any steps whatever to warn people with Stargardt's disease (who do indeed suffer from macular degeneration, and who are prime targets for products designed to prevent or slow down macular degeneration) that patients with Stargardt's disease probably would be well-advised to minimize their intake of beta-carotene, since beta-carotene is the clear and undeniable dietary ingredient that leads to the toxic A2E metabolite, which eventually will destroy the retinas of Stargardt patients and render them blind.

Zeaxanthin and lutein, which are created in plants from beta-carotene, have the chemical structures shown in FIG. 2. They are crucially important in any discussion of retinal or macular disorders, because they are the two carotenoid pigments that give the macula its yellowish color. Because of their UV-absorbing and anti-oxidant activity, they have come to be recognized as probable useful agents for helping prevent or treat most types of macular degeneration. This utility is described in U.S. Pat. No. 5,747,544 (Garnett et al 1997) and reissue patent Re-38,009 (Garnett et al 2003, which replaced U.S. Pat. No. 5,827,652, Garnett et al 1998), which are incorporated herein by reference, as though fully set forth herein. Articles that discuss zeaxanthin and lutein in human retinas include Snodderly 1995, Landrum et al 2001, Krinsky et al 2003, Semba et al 2003, and Gale et al 2003.

As indicated by FIG. 2, zeaxanthin and lutein are formed when hydroxy groups (—OH) are added to the end rings of beta-carotene. The presence of even a single oxygen atom in a carotenoid molecule causes the carotenoid to be classified as a “xanthophyll” (sometimes referred to as a xanthin or xanthine compound). Zeaxanthin and lutein are xanthophylls. They are isomers of each other, and the only difference between them is the placement of one of the double bonds, in one of the two end rings, as indicated by the arrow in FIG. 2. That is a subtle difference; most people will not even notice it, when examining those two chemical structures, which explains why an arrow was placed in FIG. 2, to call attention to that difference.

Lutein is the heavily dominant carotenoid, in plants. Even in plants that contain unusually high levels of zeaxanthin, such as spinach or kale, there is at least 50 times as much lutein. This heavy predominance evolved over the eons, in plants, because the epsilon ring at one end of lutein causes it to have a somewhat kinked and bent structure. That kinked structure allows lutein to fit ideally into circular “light-harvesting structures” in plant chloroplasts. Those structures help plant cells (and certain types of photosynthetic bacteria) carry out photosynthesis more efficiently. By contrast, since zeaxanthin has a straight chain with no kink near the end, it cannot fit properly into those circular “light-harvesting structures”. As a result, those circular structures use lutein, but not zeaxanthin. Zeaxanthin became involved in an alternate pathway, in which it alternates and shuttles back and forth with a different carotenoid called violaxanthin, in a day-night cycle. That cycle prevents zeaxanthin from accumulating in larger quantities.

Lutein is readily available, in bulk and at low cost, since it can be extracted in semi-pure form from the bright orange petals of marigold flowers. It has been used commercially for at least 20 years as a pigment, for poultry and farm-raised salmon. Huge marigold fields (formerly in Mexico, now mainly in China and India) are used to grow lutein-enriched strains of marigold, which can be extracted into a thick oily liquid (an “oleoresin”) by known processes. Two companies that sell purified lutein in bulk are Kemin Foods, and Cognis. Both sold it for poultry and salmonid pigments, and both have begun selling and promoting it for human eye and vision care.

By contrast, since zeaxanthin is very rare among plant sources, and since there was no good plant source that allows it to be created and extracted in bulk, concentrated zeaxanthin did not became available commercially until 2002. Although a bacterial source was identified in the early 1990's (described in U.S. Pat. Nos. 5,308,759 and 5,427,783, both invented by Gierhart), those fermentation and extraction processes were never scaled up to commercial volumes. In the 1990's, Roche Vitamins Inc. (subsequently acquired by DSM Chemicals) developed an efficient method of chemical synthesis, which led to the first commercial sales. Recently, Chrysantis (a division of Ball Horticultural Company) has announced a specially-bred line of marigolds that make high quantities of zeaxanthin, and it is approaching the market with an “all-natural” supply.

It also should be mentioned that some (but not all) forms of zeaxanthin and lutein that are created in plants are initially in ester form, in which fatty acids are coupled to the hydroxy groups on the end rings, through ester bonds. Since those ester bonds typically are broken apart, either by chemical extraction and processing steps, or inside an animal gut (mainly by esterase and lipase enzymes), in ways that release the “free” (or hydroxy, or alcohol) versions of zeaxanthin or lutein shown in FIG. 1, any such ester is regarded as a functional and nutritional equivalent of “free” zeaxanthin or lutein.

Zeaxanthin and lutein are discussed in more detail, below. As it turns out, the minor and subtle structural difference between them leads to crucially important differences in how they are handled by human retinas, and in the benefits they can offer for eyesight. Those factors are not recognized or understood by optometrists, ophthalmologists, or medical researchers; therefore, they are not conceded to be prior art in the field of medical technology and vision research, and they are discussed under the Detailed Description section, below.

However, it has been known for 20 years (Bone et al 1985) that lutein and zeaxanthin are the only two carotenoids that contribute to the yellowish color of the human macula. Accordingly, Zhao et al 2003 analyzed the “macular pigment optical density” (MPOD) of people suffering from retinitis pigmentosa, choroideremia, and Stargardt's disease, using a noninvasive laser technique called resonance Raman spectroscopy. They reported that, although the number of people with Stargardt's disease who were tested were small, such patients “usually” had lower levels or macular pigments, compared to people without known macular defects.

In addition, Sundelin et al 2001, Shaban et al 2002, and Nilsson et al 2003 reported that when various antioxidants (including lutein, zeaxanthin, and lycopene, as well as Vitamins E and/or C) were added to in vitro cell cultures containing rabbit or bovine RPE cells, the anti-oxidants helped suppress cell toxicity and the formation of lipofuscin, when the cells were challenged by factors such as abnormally high oxygen concentrations, or intense exposure to blue light. Similar results using somewhat different approaches were also reported in Wrona et al 2003 and 2004.

However, despite the research that has been done to date, there is no known adequate treatment for Stargardt's disease.

In particular, it should be noted that, in the absence of any convincing data from any clinical trials on humans, neither zeaxanthin nor lutein has been widely accepted or endorsed by skilled ophthalmologists or optometrists who actually work with and treat people who suffer from eye disorders. The typical attitude of most ophthalmologists or optometrists has been, and continues to be, “My patients can try it if they want to, and it probably won't hurt them. But I'm not going to endorse, recomrnend, or prescribe it, because I'm not convinced it's going to work. Even though there is nothing else out there that can stop the advance of macular degeneration or Stargardt's disease, I'm not going to recommend either zeaxanthin or lutein to patients with Stargardt's disease. If they want to take it, that's their decision, not mine.”

The National Eye Institute has also declined to take any steps that would help organize or advance any clinical trials of zeaxanthin. Despite explicit and repeated requests from the Applicant and certain others, the NEI managers in charge of planning the AREDS-2 trial have offered no support of any sort for testing zeaxanthin in any way that would enable it to be compared against lutein. Instead, they are planning and intending to test what is essentially a lutein preparation, containing a relatively small quantity of zeaxanthin. Regrettably, the design of that test will prevent anyone from being able to identify and evaluate how much benefit (if any) was contributed to patients with macular problems, by each of those two agents. These facts and positions are made clear from the name of the group that is helping plan the AREDS-2 trial, which is called the “Lutein/DHA Advisory Group.” These facts are made even more clear, by the official minutes of a meeting of that group, organized and conducted by officials of the National Eye Institute on May 17, 2004.

Despite that lack of acceptance among experts in this field of research, the Inventor herein remains convinced that zeaxanthin will perform substantially better than lutein, in the treatment described herein. The reasons for that belief are described below, under the Detailed Description heading.

After studying what is known about Stargardt's disease, and after studying results that have become available on the use of isotretinoin by a few Stargardt patients and the testing of isotretinoin using the ABCR −/− mouse model, the Inventor believes zeaxanthin, preferably without lutein but possibly with certain other anti-oxidants (such as Vitamins E and C and zinc), is likely to provide highly useful and synergistic benefits, if coadministered with a short-chain retinaldehyde inhibitor (such as isotretinoin, or an analog or derivative thereof), in patients who suffer from Stargardt's disease or other lipofuscin accumulation disorders.

Accordingly, one object of this invention is to disclose a treatment combination, using zeaxanthin (a natural carotenoid nutrient) combined with isotretinoin (ACCUTANE) or any other analogs, derivatives, or variants of isotretinoin that are found to be suitable for such use, for treating patients who suffer from Stargardt's disease or other retinal disorders involving lipofuscin accumulation and/or ABCR gene or protein defects.

Another object of this provisional application is to disclose a composition of matter, comprising a mixture of zeaxanthin and a drug that can inhibit short-chain dehydrogenase enzymes and/or RPE65 enzymes in retinal tissue, for treating patients who suffer from Stargardt's disease or other retinal disorders that involve lipoftiscin accumulation and/or ABCR gene or protein defects.

These and other objects of the invention will become more apparent through the following summary, drawings, and description.

### SUMMARY OF THE INVENTION

Zeaxanthin, a natural carotenoid found in healthy diets, offers a useful adjunctive agent that can improve and increase the ability of enzyme inhibitor drugs to slow down the retinal damage that occurs in people who suffer from Stargardt's disease or other lipofuscin disorders. Such enzyme inhibitor drugs include retinoid analogs that can bind to one or more enzymes that are involved in creating one or more unwanted metabolites (such as the A2E toxin, which kills retinal cells in patients who suffer from Stargardt's disease, or surplus quantities of all-trans-retinal, which normally is a natural and healthy component of the visual cycle, but which accumulates at unwanted quantities in patients who suffer from Stargardt's disease or who carry the ABCR ± genotype).

One such enzyme inhibitor drug that has been identified is isotretinoin, commonly known by the trademark ACCUTANE™. It has a 13-cis bent structure that sits at a midpoint between the all-trans and 11-cis isomers of retinal, which cycle back and forth in the vision cycle. Because its structure resembles those two natural isomers, isotretinoin will bind to at least two known retinal enzymes, known as RPE65 and “short chain dehydrogenase”, in ways that inhibit their activity, leading to lower rates of unwanted all-trans-retinal accumulation. However, isotretinoin has toxic side effects, and it did not work well in an animal model unless administered at a very heavy dosage; therefore, the people who made that discovery have launched a screening program to test other retinoid analogs, in the hope of identifying one or more compounds that will be more potent and effective in inhibiting one or more enzymes involved in vision processing.

The current invention discloses that by coadministering zeaxanthin along with isotretinoin, other retinoid analog drugs, or other vision enzyme inhibitor drugs that may be identified in the future, the efficacy and benefits of treatment Stargardt's and other patients with such enzyme inhibitor drugs can be increased, thereby providing better vision benefits while also allowing lower dosages of such enzyme inhibitor drugs (with accompanying reductions in safety risks and other unwanted side effects).

Expanded formulations or treatment regimens that include other active agents that can benefit eye and vision health in such patients are also disclosed.

### BRIEF DESCRIPTION OF THE DRAWINGS

FIG. 1, which is prior art, depicts the structures of all-trans-retinal and 11-cis-retinal, which are natural isomers that cycle back and forth in the vision cycle. It also indicates how isotretinoin (ACCUTANE), with a bend that involves the #13 carbon atom, sits at a midpoint between those two isomers, making it a non-natural analog that can bind to and inhibit certain enzymes that are involved in the vision cycle.

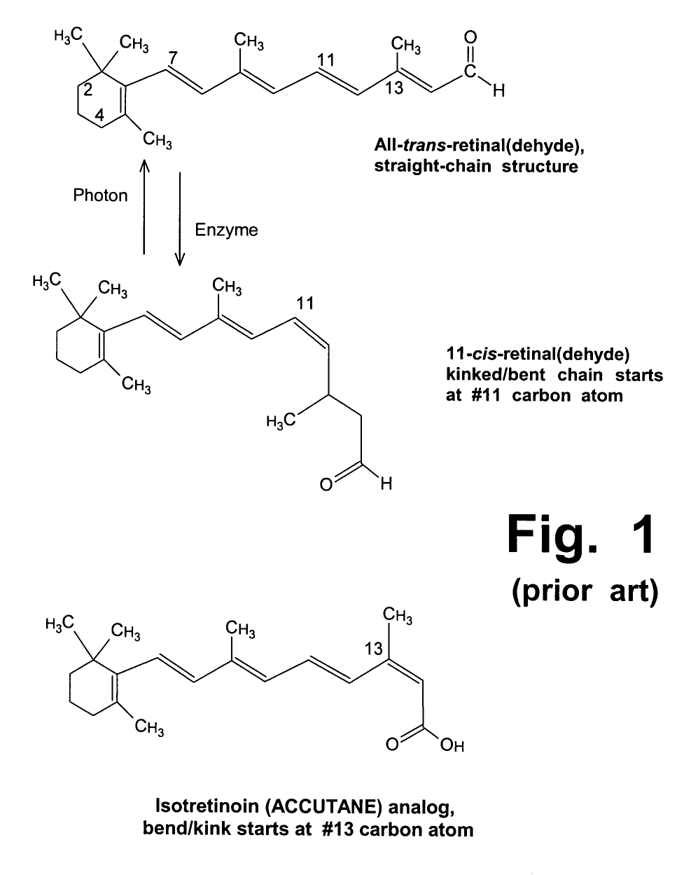

FIG. 2 depicts and compares the chemical structures of beta-carotene, lutein, and zeaxanthin.

FIG. 3 depicts the deposition and alignment of beta-carotene, lutein, and zeaxanthin in the outer membranes of animal cells.

FIG. 4 depicts a number of metabolic pathways that are likely to aggravate tissue damage and cell death in the hours and days following a treatment for “wet” macular degeneration, using a laser to activate a drug called verteporfin, which releases destructive radicals to help suppress the growth of unwanted retinal capillaries.

FIG. 5 depicts a number of ways in which zeaxanthin can suppress and reduce the toxic and destructive activities shown in FIG. 4, which otherwise would aggravate retinal cell and tissue damage in a patient suffering from “wet” macular degeneration after a laser-verteporfin treatment.

### DETAILED DESCRIPTION

As summarized above, zeaxanthin, a naturally-occurring carotenoid that is present in healthy diets, can offer a potent and highly useful adjunctive treatment for accompanying enzyme inhibitor drugs that can help slow down and prevent the accumulation of unwanted metabolites in the eyes of patients suffering from Stargardt's disease and other retinal disorders characterized by unwanted formation and/or accumulation of one or more metabolites. Such unwanted metabolites can include, for example: (i) the A2E toxin, which is gradually formed in the retinal pigmented epithelium (RPE) layers of the eyes of people who suffer from Stargardt's disease; (ii) excessive and unwanted quantities of all-trans-retinal, which promotes and aggravates the formation of the A2E toxin in Stargardt patients; and, (iii) lipofuscin and/or drusen, which are types of cellular or metabolic debris that are found in the retinas of aging people and people who suffer from various types of retinal debris.

The types of enzyme inhibitor drugs that are of interest herein include drugs that can help suppress one or more enzyme activities and pathways that are known to cause or aggravate the formation and/or accumulation of one or more such unwanted metabolites. A promising but not exclusive class of such compounds includes retinoid analogs, such as isotretinoin, commonly known by the trademark ACCUTANE™. Since isotretinoin structurally resembles both all-trans-retinal and 11-cis-retinal, and sits at a “midpoint” between those natural compounds (which play crucially important roles in the chemical cycle that enables arriving light to be converted into nerve impulses), it is able to bind to at least two known retinal enzymes, which are RPE65, and “short chain dehydrogenase”. Isotretinoin binding reactions tend to inhibit the activities of at least those two and possibly other retinal enzymes, because the enzymes cannot rapidly release the isotretinoin and go back to handling and processing normal and natural compounds.

Since isotretinoin already has been approved for treating other medical problems (mainly complexion problems, mainly among teenagers), it is of direct interest and offers an enzyme inhibitor drug that can be directly tested in humans, both with and without zeaxanthin at an appropriate dosage (such as 10 to 20 mg/day), in various classes of patients such as Stargardt's patients, and people who are over the age of about 50 and who have the ABCR ± genotype.

Alternately, researchers such as Gabriel Travis, the inventor of US patent application 2003/032,078 (Ser. No. 09/885,303), discussed in the Background section, have commenced screening programs to test and evaluate analogs, derivatives, and other variants of isotretinoin and other retinoid compounds, in an effort to identify one or more candidates that have a desired combination of efficacy and safety traits. Any such candidate drugs that are identified in that screening program or any other similar research programs is likely to perform better if accompanies by zeaxanthin as disclosed herein, and can be evaluated both with and without zeaxanthin as an adjunct, to determine whether the combination can work synergistically.

Any such usage or testing of zeaxanthin preferably should use supplement pills (which are commercially available from ZeaVision LLC under the EYE PROMISE trademark) having a known and fixed unit dosage, and that can that can be taken orally, preferably with meals. A suitable long-term dosage regimen for most people will be 10 to 20 mg/day; if desired, a higher dosage regimen can be taken for the first 2-4 weeks, to build up macular pigment levels more rapidly, especially if a patient begins a treatment regimen with relatively low levels of macular pigment, as can be determined by various types of known tests, such as flicker photometry, scanning laser ophthalmoscopy, etc.

The conclusion and belief that zeaxanthin can increase the safety, efficacy, and benefits of such enzyme inhibitor drugs is based on a combination of insights and recent findings. One set of insights arises from certain molecular, cellular, and physiological differences between zeaxanthin versus two similar carotenoids, lutein and beta-carotene, which were introduced above, and which are described in more detail below.

Additional insights arose from a fortuitous event involving an elderly acquaintance of the Applicant, who was taking zeaxanthin and who decided to have a treatment known as “photodynamic therapy”, which uses a laser to convert a drug called verteporfin into a toxin that will kill and seal newly-growing blood vessels in the retina. The patient's doctor, who is regarded as one of the world's foremost experts in treating macular degeneration, advised the patient to stop taking zeaxanthin before the laser treatment, because (in the physician's opinion) it likely would not help, and it might interfere with the laser treatment. That advice was entirely consistent with the packaging information that accompanies verteporfin, which states that carotenoids should be avoided before treatment, since they may interfere with the treatment. However, against the doctor's advice, the patient decided to continue taking zeaxanthin, and the results of the laser treatment were much better than anyone had expected.

Those insights are described in more detail below.

Zeaxanthin Chemistry, Physiology, and Deposition

As mentioned above and in the Background section, one set of insights that led to this invention arose from detailed and intensive studies, over numerous years, into carotenoid chemistry, retinal physiology, and the molecular distinctions and biological differences between zeaxanthin versus two other carotenoids of interest, beta-carotene and lutein.

Although most of the points below can be either gleaned or implied from a study of the scientific and medical literature, many of these facts are obscure, and are known mainly to specialists in plant biology or carotenoid chemistry, which are not the relevant fields of science or medicine for treating retinal diseases such as Stargardt's disease. Many of the facts mentioned below are not known to, and have not been noticed, recognized, or appreciated by, ophthalmologists, optometrists, or vision researchers. Indeed, some of the facts discussed below have been “glossed over” and deliberately obscured, in press releases, advertising, and other publications by companies that are by selling lutein, and that do not want ophthalmologists, optometrists, vision researchers, or elderly purchasers to know or understand why zeaxanthin actually is better and more effective than lutein.

Accordingly, the facts below are not conceded to be known and understood, as prior art, among experts who study or work with medical treatments for Stargardt's disease or other retinal diseases.

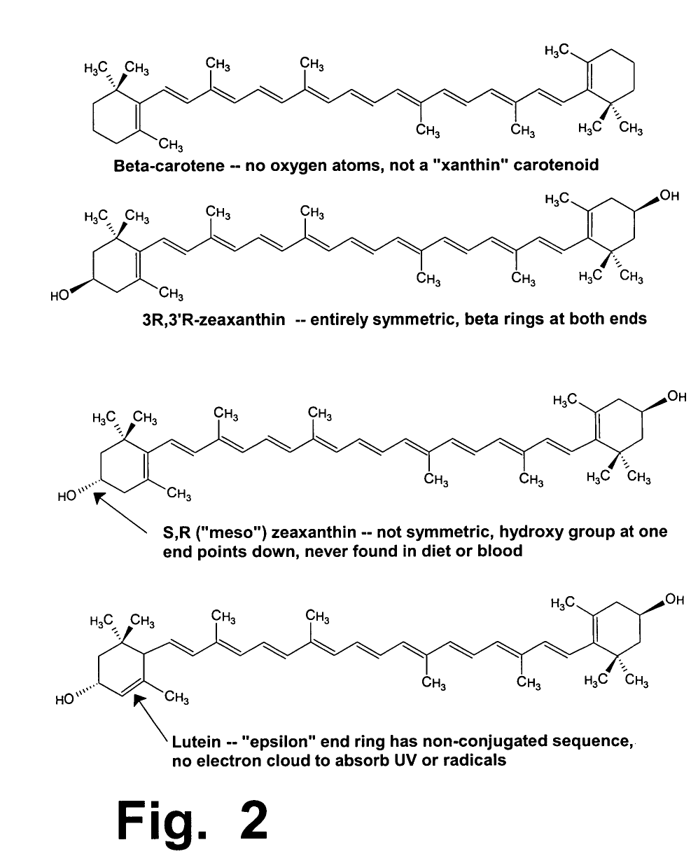

The structures of three carotenoids that are relevant to eye care formulations are shown in FIG. 2. Several factors in these structures that need to be pointed out and explained, because their significance is not readily apparent, include the following:

1. In the straight chain portion (between the two “end rings”) of all three carotenoids shown in FIG. 2, the double bonds alternate with single bonds. That pattern of alternating single and double bonds is called “conjugation”. It is crucially important, in all carotenoids, because when a series of single and double bonds are conjugated, the electrons that form bonds between adjacent atoms do not remain attached to specific atoms, and are not “pinned down” to specific bonds between atoms. Instead, those electrons become mobile, and they form an “electron cloud” that covers and surrounds the molecule. This same type of electron cloud also surrounds and stabilizes benzene rings, and other aromatic molecules.

2. The conjugated electron cloud that surrounds parts of a carotenoid molecule is crucially important, because it leads to remarkable results.

First, when a carotenoid is hit by ultraviolet light, the carotenoid will not break. Instead, the electron cloud is able to flex and yield, in a way that cushions and absorbs the blow. This is comparable to someone hitting a wooden board, and a rubber tire, with a sledgehammer. The board will break, because it cannot bend or deflect. However, a rubber tire will not break, because it can flex and yield in a way that allows it to absorb the force of the blow. In a similar manner, when destructive UV radiation hits a carotenoid molecule, the destructive power of that radiation is absorbed, by the flexible, movable, adaptable, conjugated electron cloud. This prevents the UV photon from attacking and damaging other crucial molecules, such as strands of protein or DNA. By absorbing UV radiation, carotenoids protect DNA, proteins, lipids that form cell membranes, and other crucially important molecules in cells. This is the main reason why carotenoids evolved as crucially important molecules in plants, and in microbes that must be able to grow in locations that expose them to direct sunlight for hours each day.

3. In addition to protecting molecules and cells against UV radiation, carotenoids are also anti-oxidants. This means they can neutralize, absorb, and quench unstable and destructive oxygen radicals. As discussed above, radicals are unstable and destructive, because they have unpaired electrons. The presence of an entire flexible and movable cloud of electrons, surrounding a carotenoid molecule, gives it the ability either to (i) receive and accept an extra (unpaired) electron from a “triplet” radical that has an extra electron, or (ii) donate an electron to a “singlet” radical that needs one more electron to become stable.

4. The UV-absorbing and anti-oxidant properties that arise directly from “conjugated electron clouds” can explain why it is highly important that zeaxanthin has a longer and better conjugated electron cloud, than lutein. The only difference between them is in the location of a certain double bond, in one of their two end rings, as shown by the arrow pointing to the “epsilon” end ring of lutein, in FIG. 2.

5. Zeaxanthin has two identical end rings, both with “beta” structures. The double bonds in both of those “beta” rings are positioned in a way that matches, sustains, and extends the alternating double-single bond sequence, in the straight-chain portion of the molecule. Therefore, a protective conjugated electron cloud extends out over a portion of both of zeaxanthin's two “beta” end rings.

6. By contrast, lutein has only one “beta” end ring, while its other end ring has an “epsilon” structure. In lutein's “epsilon” ring, the double bond is misplaced, in a way that does not extend, support, or allow conjugation. Therefore, lutein has no UV-protective, radical-quenching electron cloud over one of its two end rings.

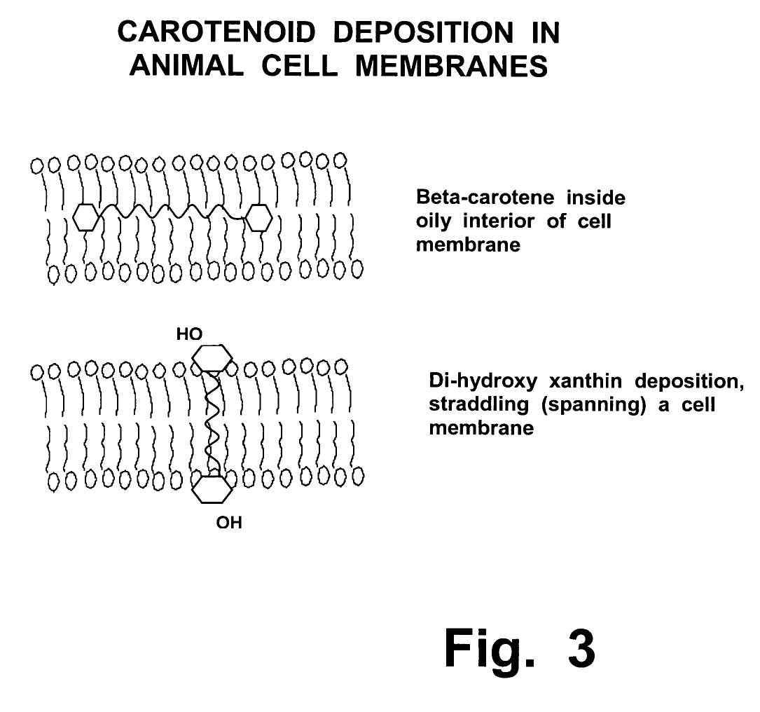

7. This defect in the “epsilon” end ring of lutein (which is not covered at all by conjugated, UV-absorbing, radical-quenching conjugated electron cloud) becomes even more important, because of how lutein and zeaxanthin are deposited and positioned in an animal cell. As illustrated in FIG. 3, both molecules are positioned in a way that causes them to straddle the thickness (width) of an animal cell's outer membrane. This positioning results from how carotenoids interact with animal cell membranes. As described in any textbook on cells or physiology, cell membranes in animals are formed from phospholipids, which are long molecules with a “head” that is hydrophilic (water-soluble), bonded to a “tail” that is oily and hydrophobic. When placed in water, these molecules will spontaneously line up in “bilayer” spheres, with the outer and inner surfaces covered by the water-soluble “heads”. The oily “tails” try to minimize their contact with water, so they line up in a way that forms an oily center layer, which fills up the interior of the cell membrane.

Zeaxanthin and lutein both have oily and hydrophobic straight-chain portions, connecting their end rings. This straight-chain portion, in both molecules, will come to rest inside the oily center layer inside an animal cell membrane.

By contrast, the end rings of zeaxanthin and lutein have hydroxy groups attached to them. These hydroxy groups are hydrophilic, and they seek contact with watery fluids. As a result, zeaxanthin and lutein will be positioned in a way that causes the tips of their end rings to extend and protrude outwardly, from both the interior and exterior surfaces of cell membranes. It is no mere coincidence that zeaxanthin and lutein have molecular lengths that are perfectly suited for that type of membrane “straddling” (or spanning) position, with only parts of their end rings sticking out and accessible. Plants and animals co-evolved over the eons, and zeaxanthin and lutein were selected for that type of membrane-straddling positioning, in animal cells, because they have exactly the right lengths.

8. The “straddling” or “spanning” orientation of zeaxanthin and lutein, in animal cell membranes, explains why the minor structural difference between their end rings becomes crucially important. Zeaxanthin's conjugated electron cloud covers part of both of its end rings; therefore, zeaxanthin it can extend a UV-protective, radical-quenching electron cloud out beyond both surfaces of an animal cell membrane.

By contrast, as noted above, lutein's “epsilon” end ring has a misplaced double bond, which disrupts and prevents the electron cloud from covering part of its epsilon end ring. Since the electron cloud is crucial for absorbing and protecting against UV radiation and destructive radicals, that apparently minor structural difference between zeaxanthin and lutein becomes crucially important, in determining and governing how they actually perform, after they are eaten by animals.

9. Another factor also should be noted, to distinguish zeaxanthin from lutein. Zeaxanthin is perfectly symmetrical, end-to-end. Both of its ends are entirely identical, in all respects. It does not matter which end of a zeaxanthin molecule happens to be “grabbed” by an enzyme that will insert the zeaxanthin molecule into an animal cell membrane.

By contrast, lutein is not symmetrical. Its two ends are different from each other.

It is not fully known how lutein's lack of symmetry affects its placement, in animal cell membranes. For example, it is not known whether lutein's beta ring (with a partial protective cloud) is placed exclusively or predominantly on either the exterior surfaces or interior surfaces of animal cell membranes, or whether that placement is essentially random and evenly divided.

However, based on other factors and observations that are known, it is clear that lutein's lack of symmetry cannot be helpful, or beneficial, when animal cells or tissues attempt to use it. For example, it is well known that zeaxanthin is deposited preferentially into the crucially-important center of the macula, lutein is deposited at low concentrations in the center of the macula, and at high concentrations around the less-important outer periphery.

10. Indeed, it is known that the human macula even attempts to convert lutein into zeaxanthin, using processes that are not fully understood. However, that conversion process cannot convert lutein into the normal form of zeaxanthin found in nature, which is the 3R,3'R stereoisomer. Instead, conversion inside retinal tissues converts lutein into a different and highly unusual stereoisomer of zeaxanthin. One end ring has the conventional “R” configuration; however, the second end ring has an unnatural “S” configuration that is not found in any dietary sources, or in human blood. The S—R isomer is often called meso-zeaxanthin, and it is discussed below.

11. Yet another factor that deserves mention and an explanation is this. Lutein is much more abundant than zeaxanthin, in plants. Even in dark green vegetables with relatively high natural zeaxanthin content (such as spinach and kale), lutein is present at concentrations that range from at least 20 to more than 100 times greater than zeaxanthin. Since one of lutein's end rings is exactly the same as zeaxanthin's end rings, that is a curious and unusual fact; clearly, any plant cell has the necessary equipment to make zeaxanthin, so why don't plant cells make more zeaxanthin?

The answer is known, but only by botanists and a few other specialists. The positioning of the non-conjugated double bond in lutein's epsilon ring gives lutein a slightly “kinked” (or bent) configuration, near that end of the molecule. That kinked and bent structure allows lutein to fit into circular “light-harvesting” structures that are found in chloroplasts, which carry out photosynthesis in plants. Because chloroplasts and their circular “light-harvesting” structures are crucially important in photosynthesis, lutein is much better than zeaxanthin, at helping plants carry out photosynthesis. This explains why plants evolved in ways that heavily favor the production of lutein, over zeaxanthin.

As mentioned above, zeaxanthin developed a different and minor role, in photosynthesis. As part of the day-night cycle of plant metabolism, zeaxanthin is shuttled and cycled back and forth into a different carotenoid called violaxanthin. This daily cycle effectively gets rid of most of the zeaxanthin in a plant, each day, as part of the cycle, by converting it into something else. This further inhibits any accumulation of zeaxanthin in significant quantities.

However, photosynthesis does not occur in animals. Simply and bluntly, animal cells do not have or use chloroplasts, and they do not have or use any circular light-harvesting structures. As a result, lutein has no particular advantages, after it has been eaten by an animal. Rather than allowing it to fit into circular light-harvesting structures, lutein's lack of end-to-end symmetry, and the kinked/bent attachment of its epsilon ring to its straight chain, become serious problems, rather than advantages, once it has been eaten by an animal. Those problems hinder lutein's ability to fit properly into animal cell membranes, and they hinder, reduce, and limit the protective benefits that lutein can provide for animal cells.

For all of the reasons described above, zeaxanthin is believed by the Inventor herein to be substantially better than lutein, in preventing or treating problems that occur in retinas. All of the information currently known tends to support the belief and conclusion that: (i) the human macula needs and wants zeaxanthin; and, (ii) if it cannot get enough zeaxanthin, it will use lutein instead, since lutein is the “closest cousin”, and the best available substitute.

Carotenoid Benefits Beyond Protection from UV and Radicals

In addition to all of the foregoing factors, the Inventor herein also has noticed a number of correlations and previously unconnected data points that tend to suggest that zeaxanthin may also be able to help suppress and control various inflammatory steps and pathways and tissue and cell stresses, in various additional ways that have not been previously recognized or correlated.

One of the factors that led the Inventor to recognize that a carotenoid such as zeaxanthin is both (i) a crucial ingredient that is essential for eye health, and (ii) an “anchor” ingredient that can enable other useful agents to work more effectively and in a synergistic manner, was the gradual realization, which arose over a span of more than a decade of reading thousands of articles, abstracts, and patents on carotenoids, of how many different roles carotenoids can play. In particular, carotenoids can play any or all of several different roles that extend above and beyond their well-known roles in absorbing ultraviolet radiation and quenching oxygen radicals.

Since most medical researchers and ophthalmologists apparently are not aware of the activities and factors listed below, or have not yet recognized how these numerous contributing activities and factors cumulatively enable zeaxanthin to provide a remarkable range of benefits to people suffering from eye problems, a numbered list is provided below which briefly touches on half a dozen lesser-known activities of carotenoids.

One of the factors that has caused these activities to be overlooked and ignored, by medical researchers, is that each of these activities can generally be described as offering only mild, weak, and partial levels of benefit. Therefore, when it comes to matters such as designing, organizing, and paying for clinical trials to prove that these benefits can be important, these potential contributing factors fall into a highly doubtful and unreliable zone, where they do and cannot not receive serious attention.

These factors are aggravated by certain types of biases that are built into clinical trials, including factors that center around what is called the “standard of care” for any particular type of disease or disorder that is being tested. Briefly, the “standard of care” issue, which arises in designing and conducting clinical trials, generally means that it is unethical, and often even illegal in ways that can lead to lawsuits and huge damage awards, for a company to withhold from patients a treatment that is known to be effective in treating a certain type of disorder that such patients may be suffering from.

As an illustration of this doctrine, it would be effectively'impossible to carry out a human clinical trial to prove that carotenoids have a relatively low yet beneficial level of activity in helping prevent or control ocular inflammation in humans, because there are other known treatments (mainly involving anti-inflammatory steroids) that are much more targeted and potent, in treating inflammatory problems. To properly test carotenoids for anti-inflammatory effects in humans, and in order to establish useful comparative data from untreated subjects, the best known treatments (i.e., steroids) would need to be withheld from all patients being tested, including “control” patients who would receive nothing but ineffective placebos. For reasons that should be apparent to anyone who works in this area, withholding a known and truly useful and effective treatment, even from “control” patients who would receive no comparable substitute, would be totally unethical, and improper. This effectively makes it impossible to carry out a clinical trial to prove that a certain carotenoid can provide mild but potentially useful benefits against problems such as inflammatory eye disorders, when other agents are already known to be effective in treating those particular problems.

For those and other reasons, physicians, scientists, and other experts regard the factors listed below as being unproven and unreliable, without sufficient support to extrapolate any data from cell culture or animal tests to actual human medicine. Therefore, in the consensus view of most physicians, scientists, and other experts, the beliefs, conclusions, and recommendations set forth below, no matter how sincere they may be, are not adequate to support medical recommendations and prescriptions, by physicians who must diagnose and treat patients suffering from macular degeneration or other eye or vision problems.

It also should be noted that various different articles describing apparently unconnected aspects of carotenoids gradually accumulated, in the overall understanding and perspective of the Inventor herein, until they led to an insight and recognition that has never been suggested or addressed in any prior art. Accordingly, the information below, on a number of relatively weak activities of carotenoids, is regarded as part of this invention, and it is suggested and taught herein that these factors, taken together, must be combined and connected into larger cohesive framework that merits serious and careful attention by physicians, ophthalmologists, and optometrists.

Accordingly, the following factors, all of which led to a specific insight that supports and substantially contributes to this invention, need to be recognized and considered:

(1) Carotenoids have a mild ability to help control and reduce inflammation, as described in articles such as Ohgami et al 2003, Lee et al 2003, Gonzalez et al 2003, Ford et al 2003, and van Herpen-Broekmans et al 2004. Although their effects in this area are not as potent as anti-inflammatory steroids, these effects may nevertheless become important, in significant numbers of patients suffering from eye disorders, because any inflammation that involves or affects either or both eyes is an important threat and risk factor, and can lead to serious and even severe problems, including blindness.

Even a relatively slight episode of inflammation, if it directly affects either or both eyes, can permanently damage the eyesight, if the inflammation leads to increased fluid pressure involving the vitreous humor (i.e., the jelly-like clear liquid between the lens and the retina). Except in the small macular region at the center of the retina, the capillaries that serve the retina actually sit on the front surface of the retina, where they are directly exposed to fluid pressures, rather than being embedded within a structural layer behind the layer of neurons and photoreceptors (this arrangement, with capillaries sitting on the front side of the retina, is curious and counter-intuitive; it can be explained partly by evolution, and partly by the fact that except for the macular region, the remainder of the retina actually generates only coarse-resolution vision, both to reduce the number of incoming nerve signals that the brain must process in order to generate coherent vision, and to reduce the load of rod and cone receptors that must be replaced as they rapidly wear out.

Because of the “anterior” (front-surface) placement of the arteries and capillaries that serve most of the retina, if fluid pressures increase to elevated levels in the clear fluid (vitreous humor) that fills the eye, those elevated pressures will press directly against the surfaces of the retinal capillaries. Since capillary walls must be extremely thin (to promote rapid exchange of oxygen, nutrients, and metabolites), they cannot resist and push back, if elevated fluid pressures are pressed against the capillary surfaces. Therefore, following basic principles of fluid flow, even a slight elevation in the fluid pressure of the vitreous humor, inside an eye, can cause a significant portion of the blood that normally flows through retinal capillaries to be diverted. The blood supply that the retina needs will simply take different routes, elsewhere in the body, at other locations where the capillaries are not being squeezed and compressed.

This mechanism explains why glaucoma will cause blindness if not treated. Glaucoma is actually the name given to an entire class of eye diseases that share a common trait: they involve elevated fluid pressures inside the eye. This pressure elevation can be caused by any of several factors (such as the secretion of too much fluid by certain types of eye tissues, factors that hinder drainage and flow through the drainage ducts, etc.). Regardless of the cause, any disorder that involves chronic elevated pressure inside the eye is called glaucoma, and it will lead to blindness, since elevated pressures will hinder the flow of blood through the retinal capillaries.

If elevated fluid pressures inside the eye are caused by inflammation that arises due to an injury or infection, the pressure increase may not be chronic or permanent, but it may last for days or weeks. That span of time is more than long enough to inflict severe, and permanent, damage on the retina.

Therefore, the ability of carotenoids to help reduce and control inflammation, even if that beneficial activity is only mild and weak, when compared to potent drugs such as steroids, may be extremely helpful, and even crucially important, in protecting eyes and vision against permanent damage caused by decreased retinal blood flow, caused by inflammation due to an injury or infection.

It also must be recognized that anti-inflammatory steroids cannot be given to patients for long periods of time, without causing serious side effects (as can be observed in patients who must take steroids for extended periods, due to diseases such as lupus). Therefore, even though anti-inflammatory steroids are highly useful for treating acute inflammation following an infection or injury, they are not useful or desirable for most types of long-term use. By contrast, zeaxanthin and other carotenoids can and should be a part of the daily diet for an entire lifetime.

(2) Carotenoids also have a mild yet potentially helpful ability to prevent and reduce sclerosis, as described in articles such as Carpenter et al 1997. “Sclerosis” refers to hardening, stiffening, and loss of flexibility. As an illustration, arterio-sclerosis refers to hardening of the arteries.

In the eyes, sclerosis and loss of flexibility can arise not just in blood vessels, but also in certain layers and structures of the eye, especially if substantial quantities of drusen, lipofuscin, and other debris accumulate in those layers and structures. In addition to rendering the eye less able to focus on objects at varying distances, this loss of flexibility can damage certain membranes, such as the Bruch's membrane, a crucially important layer behind the retina. Therefore, by helping prevent and reduce sclerosis, even if only mildly, zeaxanthin and other carotenoids can help protect eye health and good vision.

(3) Carotenoids also have mild yet potentially useful levels of activity in controlling and regulating angiogenesis (i.e., the formation of new blood vessels), because of their ability to help suppress various angiogenic hormones and cytokines. This activity is described in articles such as Armstrong et al 1998, and Chew et al 2003. Since the formation of new blood vessels can lead to severe problems and blindness in “wet” or “exudative” macular degeneration, this activity of zeaxanthin and other carotenoids may offer significant advantages, not just in treating wet macular degeneration, but in helping to prevent it in the first place.

(4) As described in articles such as Chew et al 2003, carotenoids have mild yet potentially useful levels of activity in helping to support and stabilize mitochondria, thereby helping to suppress cell death caused by the process of “apoptosis”. Mitochondria are small organelles; dozens or hundreds of them are contained in each and every cell. They are the “furnaces” of a cell, where an energy-related process called “oxidative phosphorylation” occurs. They also are the “central executioners” of cells, which govern the process that allows aging cells to be rapidly killed and then digested, by certain types of killer cells, so that their building blocks can be recycled back into new and vigorous cells. The “programmed cell death” caused by apoptosis normally does not occur in adult neurons, since neurons cannot be replaced. However, under conditions of severe stress, neurons can begin falling into that lethal pathway, leading to the deaths of crucially important cells that cannot be replaced (and also leading, in many cases, to even more stress being placed on surrounding cells and tissues). If zeaxanthin or other carotenoids can help stabilize mitochondria, in ways that can help prevent the loss of even some of those neurons, it would offer potentially very important benefits.

(5) Carotenoids have mild yet potentially useful levels of activity in helping regulate and control certain actions of the immune system, as described in articles such as Walrand et al 2004 and Carpenter et al 1997. These activities may be manifested in ways that relate to inflammation, suppression of dendritic killer cells that otherwise might trigger and carry out apoptosis too soon, etc.; however, this type of mild and subtle contribution to proper regulation of the immune system can also be manifested in other potentially useful ways, as well. Accordingly, this factor should be noted, and kept in mind.

It must also be kept in mind that the five “secondary” activities of carotenoids, listed above, also act in addition to the primary activities of carotenoids, which are (1) protection against destructive ultraviolet radiation, and (2) protection against destructive oxygen radicals.

Upon recognizing that carotenoids (a class of compounds that cannot even be created by animals, and which must be ingested by animal in their diets) play at least seven different useful activities in animals, the Inventor herein began looking deeper into underlying factors and activities. The realizations that were gradually reached fit into a larger framework of study and understanding, involving eye and vision disorders. Several factors and insights which can help describe and explain that framework, and which help show how that framework can be put to good use, focus on “connecting rods” that connect different parts of the frame to each other. Four of those “connecting rods” can be summarized as follows:

(i) Oxygen radicals play roles in (or are created in increased quantities by) several different classes of problems, which may manifest in different but overlapping ways, such as in tissue inflammation and immune responses. As one illustration of this connection, elevated quantities of oxygen radicals have been shown to trigger the production of inflammation-triggering molecules called cytokines, as described in articles such as Ohgami et al 2003, Lee et al 2003, and Armstrong et al 1998, and Ford et al 2003. As an illustration of another connection, elevated quantities of oxygen radicals are produced by some types of immune cells, which use the oxygen radicals to help kill and digest microbes, as described in articles such as Walrand et al 2004;

(ii) Mitochondria are also actively and heavily involved in numerous processes that use or manipulate oxygen. As a result, oxygen radicals are generated at fairly high rates in mitochondria;

(iii) Cells have only limited numbers of signaling pathways they can use to communicate with each other; and,

(iv) Reports have indicated that people suffering from various types of eye problems also suffer from low carotenoid concentrations in their blood (as shown by tests on blood serum), and in their eyes (as shown by low levels of macular pigment, and low concentrations of zeaxanthin in the lenses of people suffering from cataracts).

Accordingly, after realizing that carotenoids may be called upon to perform a number of secondary protective activities in addition to their two primary protective activities, the Inventor reached two conclusions about carotenoids in human health, and especially in the eyes. Those conclusions can be summarized as follows:

1. If carotenoids are being asked to perform at least seven different known tasks (and possibly even more), all of which can converge and rise to levels of major importance if and when the eyes begin to suffer from serious problems and stress, then carotenoids are more likely than other types of molecules to become “stretched thin”, to a point where their concentrations will drop, and they will not be able to adequately handle all of the tasks and problems that are being pushed at them;

2. If it is possible to reduce any of the “secondary demands” that are likely to “siphon off” the desired concentrations of carotenoids in the eyes, by means such as administering other nutrients that can provide a balanced regimen that will help address and satisfy those secondary demands, then any newly-arriving carotenoids will be more likely to actually arrive at locations where they can carry out their essential primary roles and provide the most overall benefit.

Viewed from another perspective, zeaxanthin can be regarded as a form of “buffer”, in a system that is constantly trying to sustain an equilibrium, or “homeostasis”. Like other types of buffer compounds, carotenoids can respond to whatever is added to (or imposed upon) the system, in a way that usually will help the system move back toward its equilibrium (also referred to as the “set-point” of the system). However, as is well-known to chemists, once the outer limits and maximum capacity of a buffering system has been reached, addition of even a slight quantity of additional stress (such as an acid or alkali) can cause major swings and upheavals.

Accordingly, if a “buffering system” that is provided by carotenoids (and especially by zeaxanthin) in a human retina has already been stretched to its limit, by a combination of multiple competing demands, then that protective “buffering system” can fail, leading to a series, cascade, or mixture of stresses and problems, all occurring at once, and all acting together, in ways that are suggested by phrases such as vicious circle, witch's brew, etc.

Accordingly, it is believed that an entire set of stresses and problems in human retinas can be addressed by providing an extra supply of: (1) zeaxanthin, which can function as a type of “buffering system” to help the retinal cells and tissues handle a variety of stresses and demands, and (2) an assortment of additional ocular nutrients that have been described elsewhere, including at least two or more nutrients selected from the following list:

(i) zinc, at a maximum daily dosage that is limited to about 15 to about 40 mg/day;

(ii) Coenzyme Q10, carnitine, and/or a glutathione boosting agent such as N-acetyl cysteine, any of which can help boost and stabilize mitochondrial functioning, to help prevent and suppress apoptotic cell death;

(iii) Vitamins C and/or E, at moderate dosages;

(iv) lipoic acid, a fatty acid that alternates back and forth between a reduced form and an oxidized form, and that can help reduce unwanted oxidation of cells and tissues;

(v) omega-3 fatty acids, such as docosa-hexaenoic acid (DHA);

(vi) taurine, also known as 2-amino-ethane-sulfonic acid;

(vii) beta-cryptoxanthin, a carotenoid that has been discovered to be present at unusually high concentrations in brain tissue;

(viii) carnosine, a dipeptide that norammly will be digested, and that is sometimes applied directly to the eyes, in the form of eyedrops; and,

(viii) one or more compounds (such as quercetin, genistein, eyc.) that can be isolated from certain types of plants (such as soybeans, bilberry, etc.), and that are fall into chemical categories that are called isoflavones, flavones, flavonoids, polyphenols, anthocyanins, phytonutrients, or phytohormones.

A second and additional insight is described below, which arose from an unexpected event that has not yet been reported or described anywhere in public.

Protective Benefits in Different Type of Eye Care

A second set of insights also led the Inventor herein to conclude that it is worth the effort and investment to explore and test zeaxanthin as an adjunctive treatment, along with ACCUTANE or similar drugs, in patients who suffer from Stargardt's disease. Those insights are described in detail in utility patent application Ser. No. 10/972,699, filed Oct. 23, 2004 by the same Inventor herein.

That discovery and invention arose from an event involving a personal acquaintance of the Inventor. That person was suffering from the “wet” or “exudative” form of macular degeneration, which involves abnormal and aggressive blood vessel growth in and behind the macula. He decided to have a treatment known as “photodynamic therapy”, which uses a laser that is shone directly into the eye of a patient who has been anesthetized. Before the laser treatment is carried out, the patient is injected with a drug called verteporfin, which binds to certain compounds in the blood that are carried to actively growing blood vessels. After a delay to give the drug enough time to enter capillaries in the retina, the laser treatment is commenced. The tuned wavelength of the laser beam triggers a chemical reaction that activates the verteporfin, in a way that converts it into a toxic radical compound. This toxin will attack the interior walls of the capillaries that contain it, causing it to kill and seal the newly-growing blood vessels.

Because of a number of factors, laser-verteporfin therapy is not highly efficient or effective. It is used only because there are no other known treatments that work any better (however, it should be noted that clinical trials of drugs that can block a hormone called vascular endothelial growth factor (VEGF) are showing promise). In a typical course of treatment, a patient will have up to five or six laser-verteporfin treatments, usually about 2 to about 4 months apart from each other. Each session can “knock back” and slow down the capillary growth somewhat, thereby slowing down the gradual degeneration or the retina into blindness. However, this type of treatment does not and cannot treat or reduce the underlying problem that caused the aggressive blood vessel growth, so it will eventually return in most cases, and the only benefit of the treatment is to slow down and delay the onset of blindness, usually by a period ranging from about 6 months, to about 2 or 3 years.

Furthermore, a number of serious problems with verteporfin treatments must be recognized, including the following.

The first problem is this: the transport mechanism that is used by verteporfin, to help it reach the unwanted and aggressively growing capillaries in and behind the retina, is not highly selective. It can “enrich” verteporfin concentrations inside aggressively growing capillaries, but the transport compounds used by verteporfin's “piggy-backing” approach are present in all circulating blood, in all capillaries throughout the entire retina, and indeed the entire body. Therefore, a laser-verteporfin treatment will also inflict some level of toxic damage to essential and healthy blood vessels and capillaries, in and around the retina.

A second problem is this: even if the verteporfin is present in the unwanted capillaries that are being targeted, the drug molecules that are converted into toxic radicals may not react immediately with those particular targeted capillary wall interiors. Because of the constant flow and travel of the blood, at least some of the toxic radicals that are created by the flash of laser radiation may be flushed out of the targeted capillaries, within the first few seconds after they are created. If this occurs, they will be carried into the receiving veins, and unwanted damage will be inflicted on those veins, and potentially on the retinal tissues they serve. Since that tissue is already under severe stress (due both to unwanted capillary growth, and to the underlying problem that initially triggered the unwanted capillary growth), that type of stress can trigger a fairly common biological response, which is to try to increase the supply of blood to the area that is under attack.

In other words, the type of damage that is inflicted on retinal tissue, by the creation of toxic radicals inside the retina, can lead to certain types of biological responses that will directly work against, and directly contradict and undercut, the initial goal of the therapy.

For these and other reasons, everyone who receives the treatment, and every specialist who administers it, will readily agree that it is not ideal, or even adequate. As mentioned above, even though it does not work very well, it is used, because it is the only known treatment that can retard the loss of vision for a few months, or hopefully a couple of years, before near-total blindness sets in.

As mentioned above, a person known to the Inventor herein was suffering from wet macular degeneration, and he decided to have a laser-verteporfin treatment. He chose to have it done by a specialist who is one of the world's foremost experts in that type of treatment, at Johns Hopkins Medical School. Because the patient knew about the Inventor's work with zeaxanthin and macular degeneration, he had been taking zeaxanthin for several months, at a dosage of 20 milligrams per day.

Prior to the laser treatment, during a diagnostic evaluation by the specialist at Johns Hopkins, the patient mentioned to the specialist that he was taking zeaxanthin. The specialist advised the patient to stop taking it, because (in the specialist's opinion) it probably would not help, and it might interfere with the laser-verteporfin treatment.

However, against his doctor's advice, the patient decided to continue taking zeaxanthin, and had the laser treatment.

In a surprising development, the results of the treatment were much better than anyone had expected.

That actual demonstration of the protective and synergistic benefits of zeaxanthin, in a setting involving a human patient in which an expert actually advised the patient against taking zeaxanthin, added to the Applicant's developing understanding of how and why zeaxanthin can perform better in the eyes than lutein, beta-carotene, or any other known carotenoid. That grasp of the subject matter, which arose gradually through years of focusing specifically and carefully on one compound, its chemistry, and its effects (rather than attempting to master all vision and ophthalmology research, as well as all carotenoid research) was combined with the insights that arose from the results seen in an actual patient, who defied and disregarded the advice he received from a world-class expert, who had told him to stop taking zeaxanthin before he received a different treatment as described above.

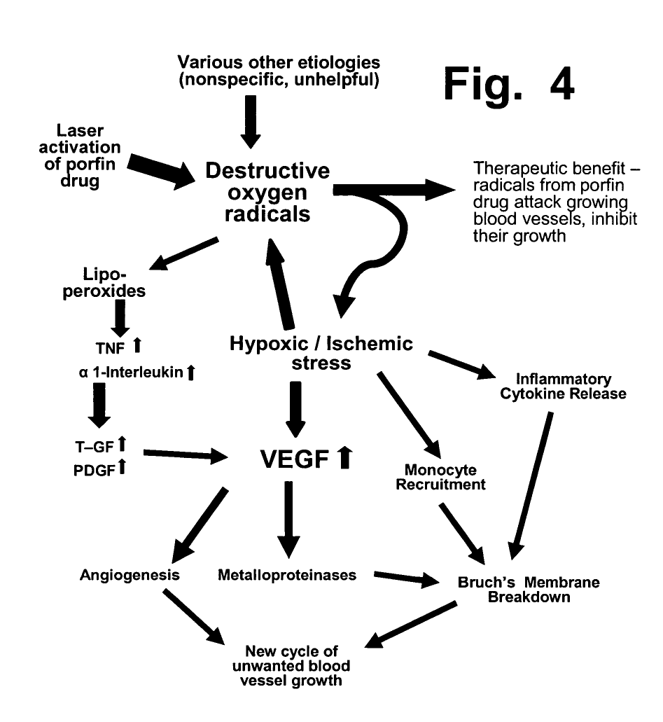

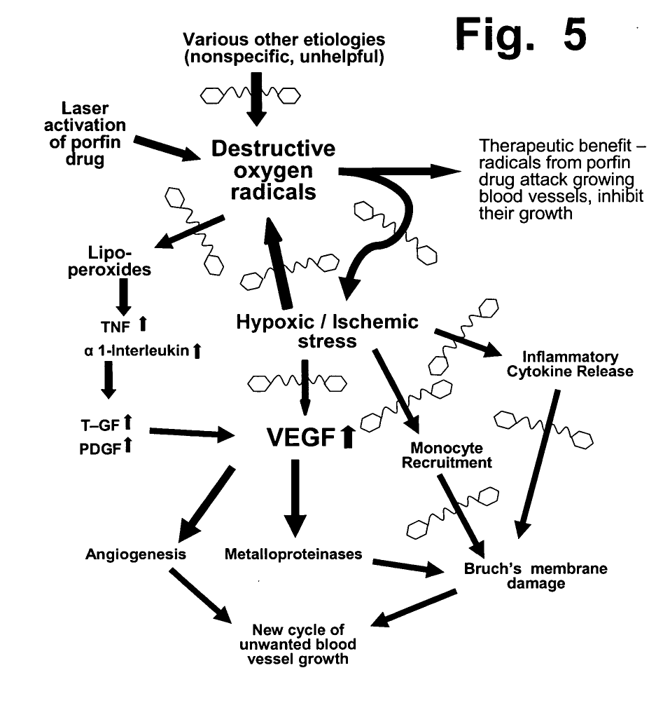

After learning about those results, the Inventor created the drawings in FIGS. 4 and 5, to help him try to understand (and explain to others) how and why a “preloading” treatment, using zeaxanthin in advance of a laser-verteporfin treatment, might be able to help improve the results of the laser-verteporfin treatment.

FIG. 4 depicts various type of potential damaging factors that may come into play, within a span of time measured in hours or days after a laser treatment session has caused the release of toxic and destructive radicals, from the verteporfin drug. These various damaging factors are likely to be present at levels that will vary substantially, among different patients who are suffering from the types of severe retinal damage that have driven them to wet macular degeneration, in which uncontrolled blood vessel growth is rapidly destroying their eyesight.

FIG. 5 uses a stylized depiction of the zeaxanthin molecule, to indicate that a number of different potentially damaging pathways, from the assortment of potential destructive pathways that may be contributing to those types of macular problems and tissue damage, might well be helped, by zeaxanthin.

Accordingly, after recognizing how many different pathological damage pathways might be suppressed and reduced, in highly useful, beneficial, and therapeutic ways, when high-dosage zeaxanthin is taken as a “pre-loading” agent for several weeks before a laser-verteporfin treatment is carried out, the Inventor herein also recognized that zeaxanthin is likely to offer similar synergistic and possibly “multi-factorial” benefits that can substantially improve the results of treatments using retinoid analogs (such as isotretinoin) in Stargardt patients, and in other patients who suffer from lipofuscin accumulation disorders and/or ABCR gene and protein defects.

The differing types and levels of benefits that may arise among different patients cannot be predicted with confidence, and patients with certain types of gene or lipofuscin disorders may receive greater benefits than patients with other disorders or defects. Nevertheless, based on everything that has been seen, read, and learned to date, it is believed that a zeaxanthin supplement regimen can and will substantially improve the outcomes of drug treatments using retinoid analogs, in at least some people who suffer from Stargardt's disease or other vision problems involving lipofuscin accumulation and/or ABCR gene or protein defects.

Coverage of Lutein by Claims

Despite the strong preference for zeaxanthin for use in formulations as disclosed herein, lutein is covered by any claims below that refer to “macular pigment”. Although it is believed that zeaxanthin will provide better results than lutein when used in combination with a retinoid analog as described herein, it should be recognized that certain companies are making large profits from lutein, and they want to continue doing so. Accordingly, those companies are acting in ways that clearly indicate that they regard zeaxanthin as a threat to their profits, regardless of whether it offers better ways to help prevent blindness. This is clearly manifested in the current plans (as of July 2005) for the AREDS-2 trial, in which only a single carotenoid formulation, with a lutein dosage five times higher than zeaxanthin is planned for testing. Those plans, if carried out, will block and prevent anyone from being able to analyze, evaluate, or quantify the differing contributions of zeaxanthin versus lutein in protecting eye health, even though the available facts strongly suggest that the human macula: (i) wants and needs zeaxanthin, (ii) uses lutein because it cannot obtain enough zeaxanthin, and (iii) even tries to convert lutein, into zeaxanthin.

Accordingly, inclusion of lutein in any “macular pigment” claims herein is intended to help create and promote a situation that will provide actual and lasting benefits for the eyes, vision, and brains of elderly consumers. If companies could avoid a set of patent claims, and make higher profits, by substituting lutein for zeaxanthin in their products even though lutein does not work as well as zeaxanthin, that situation would be counterproductive from the viewpoint of actually benefiting the public health and welfare (especially when it comes to helping grandparents get to see their grandchildren grow up).

Accordingly, lutein is covered by various claims below, not because it is equal to or interchangeable with zeaxanthin (it isn't), but to help ensure that the eye care and nutritional supplements industries are encouraged and motivated, as much as possible, to give elderly consumers the best help (and the best research) that can be provided, in the struggle against a cluster of diseases that often lead to blindness.

Benefits and Indicators in Humans and Animals

The synergistic benefits that can be achieved by combining zeaxanthin with a retinoid analog (such as isotretinoin) can be monitored and evaluated, in a human clinical trial, by measuring any or all of the following in various subpopulations of patients who suffer from Stargardt's disease, the ABCR ± genotype, or other lipofuscin accumulation disorders:

(1) the amount of A2E (a toxic metabolite) that accumulates in the RPE layer, behind the retina;

(2) the amount of lipofuscin that accumulates in and around retinal tissue;

(3) the amount of damage caused by A2E and/or lipofuscin, in or around the retina;

(4) the progressions and rates of vision loss that occur among patients being monitored;

(5) the dosages of a retinoid analog that are required to provide measurable benefits.

Since patients who suffer from fully-manifested Stargardt's disease, due to the ABCR −/− genotype and the complete absence of any functioning copies of the ABCR protein, have no other effective treatments, and since they face functional blindness as an inevitable long-term outcome of their disease, it is hoped that it will be possible to organize and carry out human clinical trials of this form of combined treatment, within the near future. Several known indicators of the progression of the disease can and should be monitored as part of any human clinical trial, principally including: (i) the appearance and concentration of A2E, a toxic component of lipofuscin, which can be easily detected due to its fluorescence, using a visual and/or photographic examination of the retina, and (ii) visual clarity, as can be measured by using various types of eye charts, printed grids, etc.

In addition, as mentioned in the Background section, an animal model of Stargardt's disease has been created, by using genetic engineering techniques to create strains of mice having “knockout” genes that cannot express properly functioning copies of the ABCR protein. These animal models show promise and potential, in testing drug-plus-zeaxanthin combinations as disclosed herein.

In planning or evaluating any animal tests, two concerns should be kept in mind. First: mice, rats, and other rodents do not have maculas, and therefore do not use lutein, zeaxanthin, or any other carotenoids as macular pigments. Second: mice, rats, and other rodents metabolize carotenoids in ways that are different, in some respects, from comparable metabolic pathways in humans and other primates.

While these concerns do not disqualify or invalidate data gathered from laboratory tests using mice or other non-primates, they need to be recognized and kept in mind. The best approach, for anyone engaged in this type of research, is to keep abreast and apprised of recent and current developments on the testing of carotenoids in rodents. Review articles that address this type of research include, for example, Gottesman et al 2001 and Cohen 2002. One of the more prominent authors in this field is J. W. Erdman Jr. His review articles include Erdman et al 1993, Lee et al 1999 (entitled, “Review of animal models in carotenoid research”), and Zaripheh et al 2002. A number of recent articles (e.g., Gonzalez et al 2003) also specifically address issues of carotenoid deposition in rodent skin.

People working in this field should also bear in mind that since carotenoids are oily and hydrophobic compounds, their testing, bioabsorption, and bioavailability can often be enhanced by strategies such as high-dosage administration, the supplemental use of permeation enhancers such as dimethylsulfoxide, and coadministration with bile salts, which are natural digestive compounds that increase the uptake (into circulating blood) of oily hydrophobic compounds. In general, any carotenoid supplements that are orally ingested should be taken with meals.

Dosages for ABCR −/− Mouse Testing

While it is premature to specify exact testing dosages for testing a combination of ACCUTANE and zeaxanthin in the ABCR −/− mouse model, such tests should optimally evaluate at least two and preferably three or more dosages of ACCUTANE, as follows:

(i) a mouse dosage that is comparable (on a milligrams per kilogram weight basis) to the highest approved dosage for humans;

(ii) the same dosage that was used by Travis et al, and that was shown to significantly reduce lipofuscin accumulation in the retinas of such mice (which was, however, roughly 40 times greater than the highest dosage allowed for use in humans); and,

(ii) at least one intermediate dosage which is at some midpoint between the two dosages specified above (such as ½, ¼, or 1/10 of the dosage used by Travis et al).

At least two, and preferably all three (or more) of the ACCUTANE dosages specified above, should be accompanied by zeaxanthin administration, for comparative purposes. To keep the costs of the tests at a reasonable level, it would appear that any initial tests would only need to use a single dietary dosage level, which can provide good and useful insights that can be used to plan any subsequent tests. The dietary dosage level recommended herein involves zeaxanthin that has been added to the “chow” that is fed to the mice, at a total concentration of 0.4% of the weight of the chow.

To help clearly evaluate the benefits of zeaxanthin compared to lutein and/or beta-carotene, the purified zeaxanthin dosage specified above can also be compared against a similar dosage of nearly-pure lutein; however, it should be kept in mind that most lutein preparations from marigolds usually contain about 2 to about 5% zeaxanthin.

Similarly, any zeaxanthin dosage can be compared against an identical beta-carotene dosage, if desired.

Thus, there has been shown and described a new and useful treatment regimen, for people who suffer from Stargardt's disease or other problems and disorders involving lipofuscin accumulation and/or ABCR gene/protein defects. Although this invention has been exemplified for purposes of illustration and description by reference to certain specific embodiments, it will be apparent to those skilled in the art that various modifications, alterations, and equivalents of the illustrated examples are possible. Any such changes which derive directly from the teachings herein, and which do not depart from the spirit and scope of the invention, are deemed to be covered by this invention.

### REFERENCES

Ben-Shabat, S, et al, “Fluorescent pigments of the retinal pigment epithelium and age-related macular degeneration,” Bioorg Med Chem Lett 2001 June 18;11(12): 1533-40

Bone, R A, et al, “Preliminary identification of the human macular pigment,” Vision Res. 25: 1531-1535 (1985)

Cohen, L A, “A review of animal model studies of tomato carotenoids, lycopene, and cancer chemoprevention,” Exp Biol Med (Maywood) 2002 November; 227(10): 864-8

Delori F C, et al, “In vivo measurement of lipofliscin in Stargardt's disease—Fundus flavimaculatus,” Invest Ophthalmol Vis Sci. 1995 October; 36(11): 2327-31

Donoso L A, et al, “Autosomal dominant Stargardt-like macular dystrophy,” Surv Ophthalmol. 2001 September-October; 46(2): 149-63

Erdman J W Jr, et al, “Absorption and transport of carotenoids,” Ann N Y Acad Sci 1993 December 31;691: 76-85

Gale C R, et al, “Lutein and zeaxanthin status and risk of age-related macular degeneration,” Invest Ophthalmol Vis Sci. 2003 June; 44(6): 2461-5

Glazer L C, et al, “Understanding the etiology of Stargardt's disease,” Ophthalmol Clin North Am. 2002 March; 15(1): 93-100, viii

Gollapalli D R, et al, “The specific binding of retinoic acid to RPE65 and approaches to the treatment of macular degeneration,” Proc Natl Acad Sci USA 2004 July 6;101(27): 10030-5

Gonzalez, S, et al, “Dietary lutein/zeaxanthin decreases UVB induced epidermal hyperproliferation and acute inflammation in hairless mice”, J Invest Dermatol 121: 399-405 (2003)

Gottesman M E, et al, “Studies of vitamin A metabolism in mouse model systems,” Bioessays 2001 May; 23(5): 409-19

Holz F G, et al, “Introduction of the lipofuscin-fluorophor A2E into the lysosomal compartment of human retinal pigment epithelial cells by coupling to LDL particles. An in vitro model of retinal pigment epithelium cell aging,” Ophthalmologe. 1999 December; 96(12): 781-5 [in German]

Koenekoop R K, “The gene for Stargardt disease, ABCA4, is a major retinal gene,” Ophthalmic Genet. 2003 June; 24(2): 75-80

Krinsky N I, et al, “Biologic mechanisms of the protective role of lutein and zeaxanthin in the eye,” Annu Rev Nutr 2003;23: 171-201

Landrum J T, et al, “Lutein, zeaxanthin, and the macular pigment,” Arch Biochem Biophys. 2001 January 1;385(1): 28-40

Lee C M, et al, “Review of animal models in carotenoid research,” J Nutr 1999 December; 129(12): 2271-7

Mata N L, et al, “Delayed dark-adaptation and lipofuscin accumulation in abcr± mice: implications for involvement of ABCR in age-related macular degeneration,” Invest Ophthalmol Vis Sci. 2001 July; 42(8): 1685-90

Mata N L, et al, “Biosynthesis of a major lipofuscin fluorophore in mice and humans with ABCR-mediated retinal and macular degeneration,” Proc Natl Acad Sci U S A. 2000 June 20;97(13): 7154-9

Nilsson S E, et al, “Aging of cultured retinal pigment epithelial cells: oxidative reactions, lipofuscin formation and blue light damage,” Doc Ophthalmol. 2003 January; 106(1): 13-6

Parish C A, et al, “Isolation and one-step preparation of A2E and iso-A2E, fluorophores from human retinal pigment epithelium,” Proc Natl Acad Sci USA 1998 December 8;95(25): 14609-13

Radu R A, et al, “Light exposure stimulates formation of A2E oxiranes in a mouse model of Stargardt's macular degeneration,” Proc Natl Acad Sci USA. 2004 April 20;101(16): 5928-33

Radu R A, et al, “Treatment with isotretinoin inhibits lipofuscin accumulation in a mouse model of recessive Stargardt's macular degeneration,” Proc Natl Acad Sci U S A. 2003 April 15;100(8): 4742-7

Schutt F, et al, “Photodamage to human RPE cells by A2-E, a retinoid component of lipofuscin” Invest Ophthalmol Vis Sci. 2000 July; 41(8): 2303-8

Semba R D, et al, “Are lutein and zeaxanthin conditionally essential nutrients for eye health?” Med Hypotheses. 2003 October; 61(4): 465-72

Shaban, H, et al, “A2E and blue light in the retina: the paradigm of age-related macular degneration,” Biol Chem 383: 537-45 (2002)

Snodderly, D. M., “Evidence for protection against age-related macular degeneration by carotenoids and antioxidant vitamins,” Am J Clin Nutr 62(Suppl): 1448S-61S (1995)

Sparrow J R, et al, “Blue light-induced apoptosis of A2E-containing RPE: involvement of caspase-3 and protection by Bcl-2,” Invest Ophthalmol Vis Sci. 2001 May; 42(6): 1356-62

Sparrow J R, et al, “Involvement of oxidative mechanisms in blue-light-induced damage to A2E-laden RPE,” Invest Ophthalmol Vis Sci. 2002 April; 43(4): 1222-7

Sparrow J R, et al, “The lipofuscin fluorophore A2E mediates blue light-induced damage to retinal pigmented epithelial cells,” Invest Ophthalmol Vis Sci. 2000 June; 41(7): 1981-9

Sparrow J R, et al, “A2E, a lipofuscin fluorophore, in human retinal pigmented epithelial cells in culture,” Invest Ophthalmol Vis Sci. 1999 November; 40(12): 2988-95

Sun H, et al, “Mechanistic studies of ABCR, the ABC transporter in photoreceptor outer segments responsible for autosomal recessive Stargardt disease,” J Bioenerg Biomembr. 2001 December; 33(6): 523-30

Sundelin S P, et al, “Lipofuscin-formation in retinal pigment epithelial cells is reduced by antioxidants,” Free Radic Biol Med. 2001 July 15;31(2): 217-25

Suter M, et al, “Age-related macular degeneration. The lipofusion component N-retinyl-N-retinylidene ethanolamine detaches proapoptotic proteins from mitochondria and induces apoptosis in mammalian retinal pigment epithelial cells,” J Biol Chem. 2000 December 15;275(50): 39625-30

van Driel MA, et al, “ABCR unites what ophthalmologists divide(s),” Ophthalmic Genet. 1998 September; 19(3): 117-22

Weng J, et al, “Insights into the function of Rim protein in photoreceptors and etiology of Stargardt's disease from the phenotype in abcr knockout mice,” Cell. 1999 July 9;98(1): 13-23

Wolf G, “Lipofuscin and macular degeneration,” Nutr Rev. 2003 October; 61(10): 342-6

Wrona M, et al, “Cooperation of antioxidants in protection against photosensitized oxidation,” Free Radic Biol Med. 2003 November 15;35(10): 1319-29

Wrona M, et al, “Zeaxanthin in combination with ascorbic acid or alpha-tocopherol protects ARPE-19 cells against photosensitized peroxidation of lipids,” Free Radic Biol Med 2004 May 1;36(9): 1094-101

Zaripheh S, et al, “Factors that influence the bioavailablity of xanthophylls,” J Nutr 2002 Mar; 132(3): 531S-534S

Zhao DY, et al, “Resonance Raman measurement of macular carotenoids in retinal, choroidal, and macular dystrophies,” Arch Ophthalmol. 2003 July; 121(7):967-72

1. A method for treating a patient suffering from a retinal disorder characterized by formation and accumulation of at least one unwanted metabolite in at least one type of eye tissue, comprising the coadministration to said patient of at least two active agents, comprising:
(i) at least one enzyme inhibitor drug, at a dosage that inhibits at least one enzyme which is involved in creating at least one unwanted metabolite; and,
(ii) at least one macular pigment carotenoid, at a dosage that provides synergistic benefits when coadministered to human patients along with said enzyme inhibitor drug.

2. The method of claim 1, wherein the macular pigment carotenoid comprises zeaxanthin.

3. The method of claim 1, wherein the enzyme inhibitor drug comprises a retinoid analog.

4. The method of claim 3, wherein the retinoid analog comprises isotretinoin.

5. A medicament for treating a patient suffering from a retinal disorder characterized by formation and accumulation of at least one unwanted metabolite in at least one type of eye tissue, comprising:
(i) at least one enzyme inhibitor drug that inhibits at least one enzyme which is involved in creating at least one unwanted metabolite; and,
(ii) at least one macular pigment carotenoid.

6. The medicament of claim 5, wherein the macular pigment carotenoid comprises zeaxanthin.

7. The medicament of claim 5, wherein the enzyme inhibitor drug comprises a retinoid analog.

8. The medicament of claim 7, wherein the retinoid analog comprises isotretinoin.
